(19) United States
(12) Patent Application Publication
(10) Pub. No.: US 2023/0352176 A1
(43) Pub. Date: Nov. 2, 2023

(54) METHODS AND SYSTEMS FOR DIAGNOSING TUMORS ON MEDICAL IMAGES

(71) Applicant: LYJ TECHNOLOGY CO., LTD., Tainan City (TW)

(72) Inventors: Lin-Yi JIANG, Tainan City (TW); Wei-Chen YEH, Taichung City (TW); Shun-Pin HUANG, Taichung City (TW)

(73) Assignee: LYJ TECHNOLOGY CO., LTD., Tainan City (TW)

(21) Appl. No.: 18/305,111

(22) Filed: Apr. 21, 2023

Related U.S. Application Data
(60) Provisional application No. 63/333,302, filed on Apr. 21, 2022.

(51) Int. Cl. G16H 50/20; G16H 70/60; G16H 80/00; G06T 7/00

(52) U.S. Cl. CPC G16H 50/20; G16H 70/60; G16H 80/00; G06T 7/0012; G06T 2207/30096

(57) ABSTRACT

The present invention relates to a novel meta-image-based tumor detection deepnet pipeline to increase the diagnosis capacity by cooperating with experts' knowledge for accurate tumor recognition in medical images.

### FIELD OF THE INVENTION

The present invention relates to methods and systems for diagnosing tumors. More particularly, the present invention relates to a method and systems for diagnosing tumors in medical images using artificial intelligent technologies.

### BACKGROUND OF THE INVENTION

Medical images from CT scanners are an important tool for doctors exploring the anatomy of the human body. Precisely locating tumor areas assists doctors to diagnose patients and even to achieve elimination of tumors by radiotherapy. Image interpretation for clinical decisions by CT is an important but onerous task, as it requires expert knowledge and large work force. Hospitals and medical researchers are naturally interested in applying advanced intelligent technologies to radiology to enhance performance and reduce incidence of false diagnosis.

The task of locating tumors in CT images is challenging for current information technologies, as certain tumors with high moisture content are imaged in pixels of gray levels similar to normal muscle or nearby organs, such as liver, pancreas, spleen, and kidney. Recent projects demonstrating momentous improvement in processing abdominal CT images mostly adopt deep network (deepnet) technologies. To improve deepnet model capacity on processing of medical images, Zhou et al. (“ACNN: A full resolution DCNN for medical image segmentation,” IEEE International Conference on Robotics and Automation (ICRA), 2020, pp. 8455-8461) and Bai et al. (“Deep interactive denoiser (DID) for X-ray computed tomography,” IEEE Transactions on Medical Imaging, Vol. 40, No. 11, pp. 2965-2975, 2021) customized the deep network structures to enhance the image resolutions. Lin et al. (“AANet: Adaptive attention network for COVID-19 detection from chest X-ray images,” IEEE Transactions on Neural Networks and Learning Systems, Vol. 32, No. 11, pp. 4781-4792, 2021) improved performance by extracting adaptive features by using deformable convolution networks. These works performing diagnosis decisions with merely medical images would result in insufficient performance on tumors due to the inherent obscurity of CT images.

In contrast, Xie et al. (“Knowledge-based collaborative deep learning for benign-malignant lung nodule classification on chest CT,” IEEE Transactions on Medical Imaging, Vol. 38, No. 4, pp. 991-1004, 2019) introduced knowledge into deepnets to classify lung nodules, where multiple sub-nets were trained to represent different views of domain knowledge, and then a classification module was used to fuse these sub-nets to diagnose lung diseases. However, the manual model creation of sub-nets and the complicated fusion process are barriers in applying the technologies to other organs. There is still a need to develop methods that can detect tumors in medical images (such as CT and X-ray images) with higher sensitivity and accuracy.

### SUMMARY OF THE INVENTION

Tumors in certain organs are shown in pixels of similar gray levels in computed-tomography (CT) medical images. This incurs low diagnosis capacity in existing computer-aided methods, such as recent deep-network (in brief, deepnet) solutions. In the present invention, a novel meta-image-based tumor detection deepnet pipeline is created, aiming at increasing diagnosis capacity by incorporating with experts' knowledge for accurate tumor recognition in medical images. The central concept is the invented meta-image model, which improves the deepnet capacity by increasing the dimension of feature space from exotic domain knowledge. Two approaches of generating meta-images from domain knowledge are presented to show its feasibility. Furthermore, for creating diagnosis models with adopted knowledge, the key is to design appropriate loss functions by counting loss values occurring in meta-images while training the tumor detection deepnet. All core mechanisms are fully formulated and elaborated with illustrative figures.

Therefore, it is an objective of the present invention to provide methods and systems for diagnosing tumors in medical images.

In a preferred embodiment of the invention, the diagnosis is made through analyzing the medical images by applying meta-image-based deepnets.

In a preferred embodiment of the invention, the medical images are CT images.

In a preferred embodiment of the invention, the medical images are X-ray images.

In a preferred embodiment of the invention, the meta-images are generated by transforming a knowledge rule (KR) by the deepnet-based approach and the analytics-based approach, wherein the deepnet-based approach comprises using deepnets to represent knowledge rules and construct a meta-image such as tumors residing in the organ region. Deepnets are used to identify the organ region and construct a meta-image. Analysis-based methods include using analytic models to represent knowledge rules and construct a meta-image. Tumors are often displayed in a specified brightness range. Analytic models are used to find pixels of a CT-image that fit the brightness range and to construct a meta-image. Other medical rules can be translated by using the two approaches. It is also acceptable to use a hybrid of the two approaches to translate knowledge rules in applications. In this manner, the human knowledge and the CT scanning data are mixed together and represented an image format. These meta-images augment features form the diagnosis deepnet so that diagnosis capacity can be improved.

In a preferred embodiment of the invention, the knowledge derived loss functions aid the deepnet optimizer to create powerful tumor detection models. The optimizer is able to tune parameters that do not meet exotic knowledge via loss function of knowledge during the model creation stage. Hence, the meta-image-based diagnosis deepnets have a high chance to obtain effective models.

Another objective of the present invention is to provide methods for processing medical images.

The present invention is described in detail in the following sections. Other characterizations, purposes and advantages of the present invention can be found in the detailed descriptions and claims of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1: Computer-aided diagnosis prompts frequently cause misjudgment of CT images created by state-of-the-art methods, since images of tumors in an organ are blurry and lack color contrast due to high moisture content. For ease of reading, an image of relatively clear tumors is chosen to illustrate the diagnostic difficulty (more confusing cases are shown in the experiments): (a) doctor labels (ground truth), (b) evaluated by a popular object-detection deep net, and (c) evaluated by the method of the present invention.

FIG. 2: Two visual examples of meta-images generated from FIG. 1(a): (a) by the deep-net-based approach and (b) by the analytics-based approach.

FIG. 3: The deepnet-based pipeline of the present invention with meta-images for diagnosing tumors with high moisture content, where the key design is to increase dimensions of feature space from transforming exotic knowledge in a uniform manner.

FIG. 4: Visualizing the implementation structures of the meta-image and the knowledge-annotated image with respect to CT image C_{w×h}.

FIG. 5: Illustrating the meta-image generation M(1)=KU-Net (C) for KR1 by using the deepnet-based approach, the structural details of which are shown in Table I.

FIG. 6: Illustrating design concepts of Lorr and Lbrr.

FIG. 7: Selected satisfied and unsatisfied cases in different meta-image-based deepnets with the YOLO backbone.

FIG. 8: Time cost of creating diagnosis models in different numbers of epochs for various deepnets.

FIG. 9: The loss value as a function of epochs in the training stage for KR1+KR2@YOLO and YOLO.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedence over any dictionary or extrinsic definition.

As utilized in accordance with the present disclosure, the following terms, unless otherwise indicated, shall be understood to have the following meanings.

The term “about” when used before a numerical designation, e.g., temperature, time, amount, concentration, and so on, including a range, indicates approximations which may vary by (+) or (−) 10%, 5% or 1%.

The term “comprising” or “comprises” is intended to mean that the systems and methods include the recited elements, but without excluding others. “Consisting essentially of” when used to define systems and methods shall mean excluding other elements of any essential significance to the combination for the stated purpose. Thus, a medicament or method consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transitional terms are within the scope of this invention.

The term “medical image” as used herein refers to images obtained from radiology, which includes but is not limited to the imaging technologies of X-ray radiography, magnetic resonance imaging, ultrasound, endoscopy, elastography, tactile imaging, thermography, medical photography, and nuclear medicine functional imaging techniques such as positron emission tomography (PET) and computed tomography (CT).

Unless otherwise required by context, singular terms shall include the plural and plural terms shall include the singular.

### I. Introduction of the Present Invention

In the present invention, a novel meta-image-based deepnet pipeline was created for the radiology industry, aiming at automatically generating useful feature sets through incorporating experts' knowledge to enhance diagnosis capacity of a tumor detection deepnet. The key design, called meta-image, is a knowledge encoding data structure that acts a knowledge carrier to enrich semantics of medical images (in technical terms, meta-images increase dimensions of feature space in the deepnet).

Two meta-image examples from two knowledge rules (KRs) are used to perform this work. The first KR is the target organ boundary constraint, wherein the corresponding meta-image increases diagnosis capacity of deepnets by avoiding false diagnosis outside the target organ. The second KR is the brightness constraint, wherein the corresponding meta-image increases diagnosis capacity by highlighting pixels satisfying the brightness region. The above idea of knowledge carriers is conceptually straightforward, but challenges exist in flexibly representing domain knowledge for being uniformly processed by current deepnets. The meta-image model is the solution to uniformly transform knowledge to the data structure that general deepnets can process. Based on the meta-image model, a meta-image-based tumor detection deepnet pipeline is then created to force general deepnet architectures to learn knowledge rules through merely modifying loss functions. In this way, the scheme of the present invention can be adopted by industries to customize the proposed pipeline to fit their business needs.

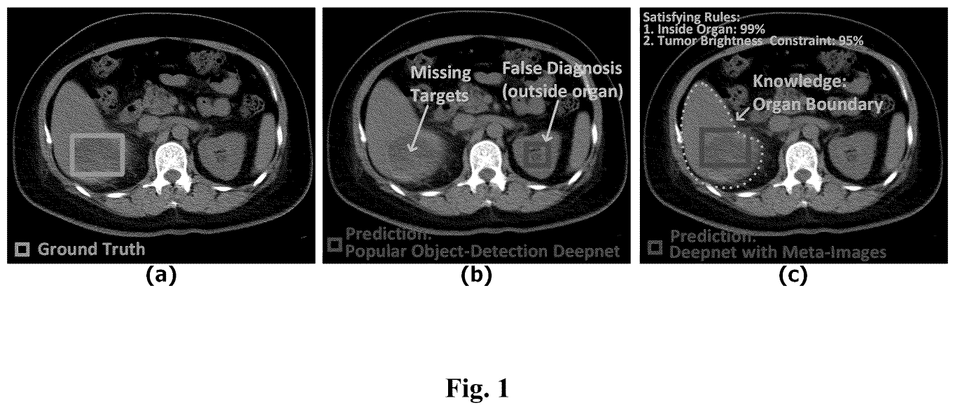

FIG. 1 gives a quick look at an example of the proposed method, where the CT image is used in diagnosing hypodense liver tumors with a well-recognized YOLO deepnet (see Redmon et al., “You Only Look Once: Unified, real-time object detection,” IEEE Conference on Computer Vision and Pattern Recognition (CVPR), pp. 779-788, 2016). FIG. 1(a) shows that tumors appear in the bottom of the liver, marked in green boxes. FIG. 1(b) shows that the trained YOLO deepnet missed tumors and incurred a false diagnosis in the neighboring organ due to their similar pixel features. These are frequently encountered problems when using most deepnets (and existing methods). Compared to existing deepnets, the method of the present invention encodes expert knowledge into meta-images, such as the liver region constraint, meaning tumors appear inside the liver boundary. Our created deepnet models are then forced to learn the knowledge. Thus, the meta-image-based deepnets have a high chance to identify indistinct tumors and eliminate false diagnosis outside the liver, as shown in FIG. 1(c).

As shown in the example section, a meta-image-based deepnet pipeline was created, and abdominal CT images from a hospital in central Taiwan were adopted for conducting a set of real-world experiments. The tumor labels were verified by doctors for confirming the practicability of meta-images generated by our approaches. Testing results explicitly reveal the superior performance of the scheme of the present invention. The present invention provides a useful reference for industrial practitioners to create intelligent medical diagnosis systems with medical images for effectively detecting obscure tumors in different organs. The contributions of the present invention are summarized as follows.
A meta-image model is created to transform domain knowledge into a form that deepnets can process.
Two meta-image generating approaches are presented to show feasibility of the meta-image model.
A meta-image-based deepnet pipeline is created to accomplish accurate tumor detection. Loss functions of integrating the meta-images of KRs into deepnets are also elaborated to increase efficacy.
A real-world case study is conducted to validate the scheme and the practicability of the present invention.

### II. Meta-Images: Machines Learn with Both Data and Knowledge

Since certain tumors are hard to identify from the perspective of computer vision, the solution of the present invention is inspired by fusing artificial intelligence and human intelligence. That is, human knowledge is affixed to medical images for enriching representative semantics. In the neural computing aspect, the scheme of the present invention expands the dimension of the feature space in deepnet by using human knowledge so that the chance of differentiating tumors from backgrounds is increased.

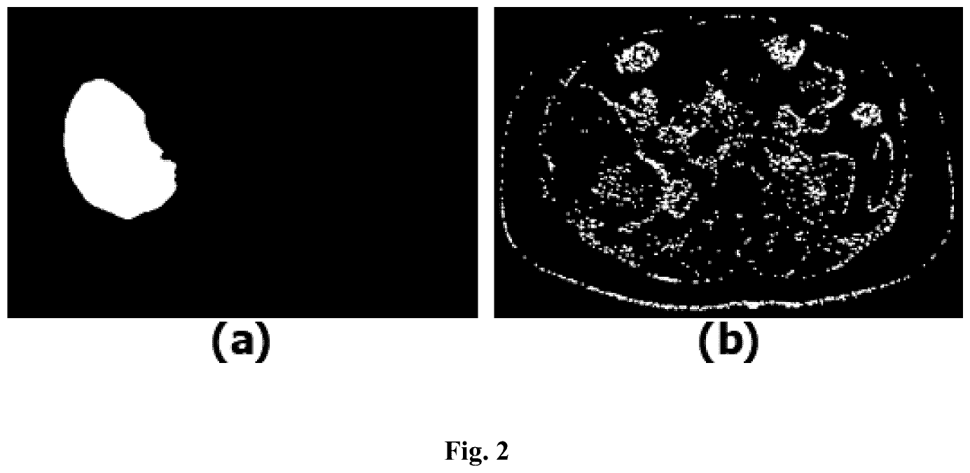

For achieving the above goal, the meta-images were invented to represent human knowledge and to be accommodated by deepnets. A meta-image is a knowledge carrier of an image format whose width and height are the same as the original medical image. For example, in the case of a knowledge rule “tumors must appear inside the liver,” the associated meta-image is an image of weighting the liver region and ignoring the non-liver area, and FIG. 2(a) is a visual representation of the meta-image.

Two ways to transform a knowledge rule (KR) into a meta-image are presented: the deepnet-based approach and the analytics-based approach, which are described as follows.

(1) Deepnet-based approach. A property of this sort of KR is that the requested targets of the rules are hard to clearly specify. Thus, deepnets are used to represent knowledge rules. An example is given below.

KR1. The tumors reside in the organ region.

Computational Translation for KR1. This rule is obviously intuitive to doctors, but the specified organ region is not fixed in a medical image. Deepnets are thus suitable to construct a meta-image for the rule, for example, U-Net (see Ronneberger et al., “U-Net: Convolutional networks for biomedical image segmentation,” International Conference on Medical Image Computing and Computer-Assisted Intervention. pp. 234-241. Springer, 2015) is used to identify the organ region in many related works. The output of the deepnet is a meta-image, where pixels inside the organ region are set to 255 (maximum value) and ones outside the organ are set to 0. The technical details will be discussed later.

(2) Analytics-based approach. The requested targets of this sort of KR can usually be explicitly expressed as calculation formulas or procedures. Thus, analytic models are used to represent knowledge rules. An example is given below.

KR2. According to doctors' experiences, tumors are often displayed in a specified brightness range, say [b⊥, b_{T}], in CT images.

Computational Translation for KR2. This rule reflects a common experience regarding how doctors quickly identify tumors in the organ. The following steps are used to generate a meta-image for KR2: (1) creating a two-dimensional matrix (i.e., meta-image) whose weight and height align the original CT image, and (2) sequentially setting the pixel value to 255 (maximum value) if brightness of the associated pixel is in the range [b⊥, b_{T}]; otherwise, set to 0. FIG. 2(b) is a visual representation of the meta-image for KR2.

Other medical rules can be translated by using the two approaches. The above two KR examples are used for illustrating the scheme of the present invention in the following sections. It is also acceptable to use a hybrid of the two approaches to translate knowledge rules in applications. In this manner, the human knowledge and the CT scanning data are combined in the image format. These meta-images augment features for the diagnosis deepnet so that diagnosis capacity can be improved. The complete computational framework in which the meta-images are applied is elaborated in the next section.

### III. The Meta-Image-Based Diagnosis Pipeline

### A. Overview

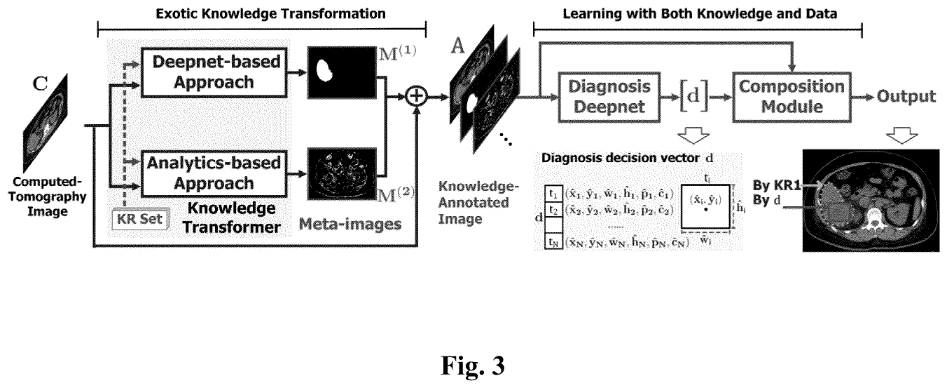

With the meta-image model described in Sec. II, FIG. 3 shows the implementable meta-image-based deepnet pipeline for tumor detection. The input is a CT image (denoted by C) and the output is a CT image with diagnosis results. There are two parts in the proposed pipeline: one is the knowledge transformer for generating meta-images from domain knowledge, and the other is a diagnosis deepnet for performing tumor detection with the knowledge-annotated image, which is a combination of the CT image and meta-images, as shown in the figure. Note that the knowledge-annotated image plays a connectivity role between the two parts in the pipeline, meaning the domain knowledge substantially influences diagnosis results in the form of meta-images.

In the first part, the knowledge transformer contains certain KR translation modules, each of which generates meta-images from knowledge rules by either the deepnet-based approach or the analytics-based approach. More specifically, for knowledge rule KR^{(k)}, the knowledge transformer generates meta-image M^{(k)} for a CT image, such as the two examples of FIG. 2 being established as M⁽¹⁾ and M⁽²⁾, respectively, in FIG. 3. After knowledge transformation, the generated meta-images and the original CT image are fused into a new knowledge-annotated image (denoted by A).

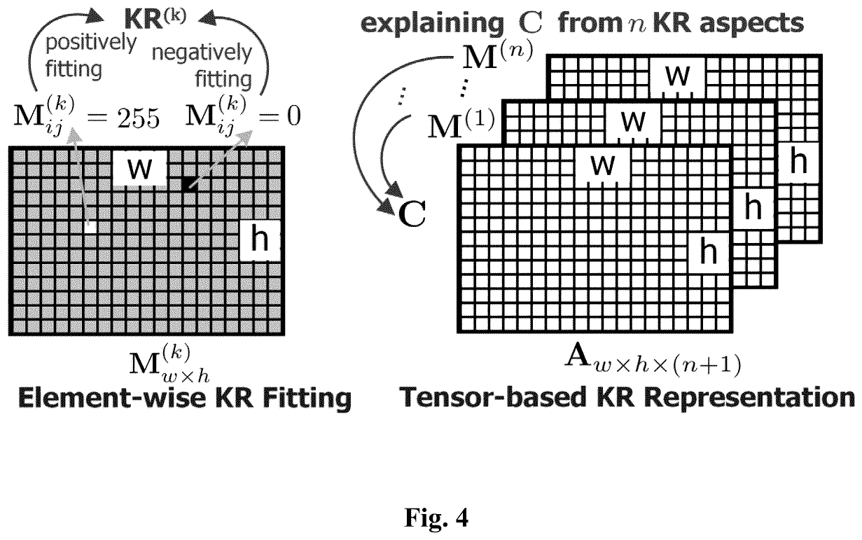

FIG. 4 elaborates structures and semantics of meta-image M^{(k)} and knowledge-annotated image A. Let the CT image C be with size w×h. Then, meta-image M^{(k)} and knowledge-annotated image A of n meta-images can be implemented by multi-dimensional arrays with size w×h and w×h×(n+1), respectively. The structures have sophisticated semantics. A meta-image represented in a matrix can be seen an element-wise KR fitting function for CT image C. For example, in the case M_{i,j}^{(k)}=255, it indicates that pixel (i,j) in C positively fits the k-th knowledge rule (KR^{(k)} in the figure); while in the case M_{i,j}^{(k)}=0, pixel (i,j) negatively fits KR^{(k)}. On the other hand, knowledge-annotated image A can be seen as a tensor-based KR representation for accommodating the original CT image C and all n specified knowledge rules. Each knowledge-rule layer consisting of M^{(k)} is a view of explaining the base layer C. The tensor formatted A is the required knowledge structure as it can be processed by most deepnets.

The second part of the pipeline contains a diagnosis deepnet and a composition module: the former detects tumors from knowledge-annotated image A and then outputs them in diagnosis decision vector d, and the latter constructs the visual diagnosis result from A and d. As this work focuses on efficacy of meta-images, the diagnosis deepnet backbone adopts the popular YOLO deepnet as the underlying network structure. The diagnosis decision vector d contains the tumor areas in rectangular boxes in the format of (center, width, height, confidence, classes), e.g., the area of i-tumor tᵢ=(\hat{x}ᵢ, ŷᵢ, ŵᵢ, ĥᵢ, \hat{p}ᵢ, ĉᵢ) in FIG. 3, whose notations are the same as YOLO for compatibility. The composition module is to render the diagnosis decision d as visual objects on the CT image, e.g., red boxes in the figure. The technical details, including meta-image generation, the deepnet architecture, and associated loss functions are separately presented in the following subsections.

### B. Generating Meta-Images and Knowledge-Annotated Image

Let the size of CT image C be w×h. Then, the size of the k-th meta-image M^{(k)} is also w×h. The general form of a meta-image generating function K can be defined as:

K: [symbol] ^{w×h}→ [symbol] ^{w×h} and M(k)=K(C),k=1,2 (1)

Functional implementations that satisfy the above definition and knowledge semantics are accepted for meta-image generators. With the discussion in the previous section, two categories of approaches, i.e., the deepnet-based and the analytics-based approaches, are used to implement K, together with two concrete examples.

Meta-images from deepnet-based approach. KR1 is used as an example to generate meta-image M(1) (shown in FIG. 3) via the U-Net; that is, M(1) can be represented as:

M⁽¹⁾=K_{U-Net}(C) (2)

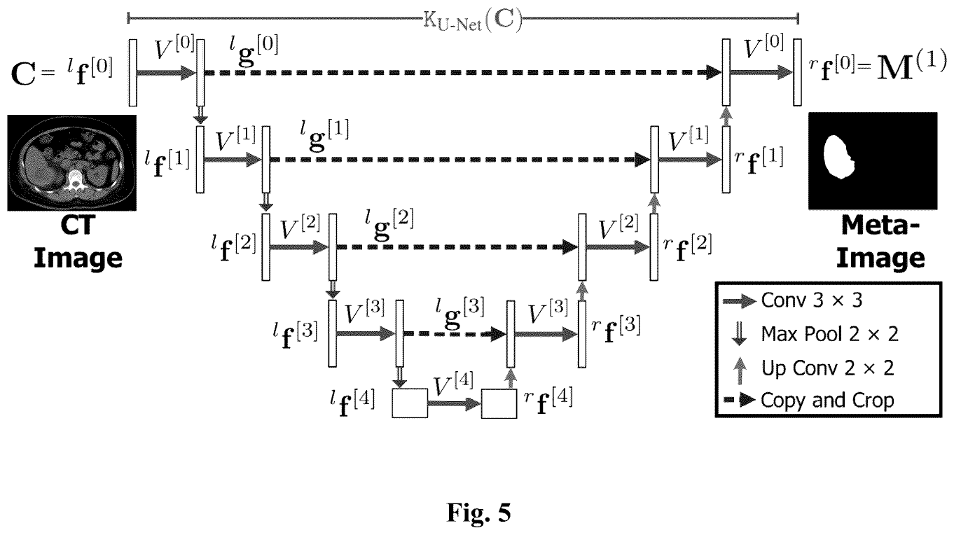

FIG. 5 visualizes the implementation of K_{U-Net}(C) to generate meta-image M⁽¹⁾ for KR1 via U-Net by weighting the organ region (liver in this case). The detailed formulation and specification of U-net architecture for generating M⁽¹⁾=K_{U-Net}(C) used in this study are described below.

Assume u levels are in the U-Net (u=5 in FIG. 5). The feature in the i-level of the left-side block (denoted as ^{l}f^{[i]}) and ^{l}f^{[0]}=C. The feature transformation operation V^{[i]}(·) consists of two convolution layers and is defined as:

V^{[i]}(^{l}f^{[i]}) [symbol] Conv(Conv(^{l}f^{[i]})),i=0, . . . ,u−1 (3)

where Conv( ) is a convolution layer used in ordinary convolutional neural networks. By applying V[i](^{l}f^{[i]}), feature ^{l}f^{[i]} is transformed into feature ^{l}g^{[i]} in the same level, i.e., ^{l}g^{[i]}=V^{[i]}(^{l}f^{[i]}). The relationship between features in two consecutive layers, i.e., ^{l}f^{[i-1]} and ^{l}f^{[i-1]}, are described by the feature-reducing function ^{l}F^{[i]}, which is defined as:

^{l}f^{[i]}=^{l}F^{[i]}(^{l}f^{[i-1]}) [symbol] MaxPool(V[i](^{l}f^{[i]})) (4)

where MaxPool( ) is a maximum-pooling layer used in ordinary convolutional neural networks. On the right-hand side of the U-Net, the feature ^{r}f^{[i]} is transformed by V on a mixture feature of concatenating features from left-hand side of the same level and from the previous level in the right-hand side, and is expressed as

$${}^{r} {f}^{[ i ]} = \{ \begin{matrix}{𝓋}^{\text{?}} ( Concat ( {}^{\text{?}} {g}^{\text{?}} , Up Conv ( {}^{\text{?}} {f}^{\text{?}} ) ) ) , & if i = 0 , … , u - 2 \\ {𝓋}^{( s )} ( {}^{\text{?}} {f}^{\text{?}} ) , & if i = u - 1\end{matrix} . \qquad ( 5 )$$

$$\text{?} \text{indicates text missing or illegible when filed}$$

where UpPool( ) is an up-convolution layer used in ordinary convolutional neural networks and Concat( ) performs a concatenation to input features. The detailed network specification is shown in Table I below.

TABLE I
Network structure of modified U-Net for KR1.

| | | | | | |
|---|---|---|---|---|---|
| Type | #(Kernels) | Size | Stride | Padding | Output Size |

Left-hand-side Block

| | | | | | |
|---|---|---|---|---|---|
| convolution | 64 | 3 × 3 | [1, 1] | same | 512 × 512 |
| convolution | 64 | 3 × 3 | [1, 1] | same | 512 × 512 |
| max pooling | | 2 × 2 | [2, 2] | valid | 256 × 256 |
| convolution | 128 | 3 × 3 | [1, 1] | same | 256 × 256 |
| convolution | 128 | 3 × 3 | [1, 1] | same | 128 × 128 |
| max pooling | | 2 × 2 | [2, 2] | valid | 128 × 128 |
| convolution | 256 | 3 × 3 | [1, 1] | same | 64 × 64 |
| convolution | 256 | 3 × 3 | [1, 1] | same | 64 × 64 |
| max pooling | | 2 × 2 | [2, 2] | valid | 32 × 32 |
| convolution | 512 | 3 × 3 | [1, 1] | same | 32 × 32 |
| convolution | 512 | 3 × 3 | [1, 1] | same | 64 × 64 |
| max pooling | | 2 × 2 | [2, 2] | valid | 64 × 64 |

Bottleneck Block

| | | | | | |
|---|---|---|---|---|---|
| convolution | 1024 | 3 × 3 | [1, 1] | same | 64 × 64 |
| convolution | 1024 | 3 × 3 | [1, 1] | same | 64 × 64 |

Right-hand-side Block

| | | | | | |
|---|---|---|---|---|---|
| up convolution | 512 | 3 × 3 | [2, 2] | same | 128 × 128 |
| convolution | 512 | 3 × 3 | [1, 1] | same | 128 × 128 |
| convolution | 512 | 3 × 3 | [1, 1] | same | 128 × 128 |
| up convolution | 256 | 3 × 3 | [2, 2] | same | 128 × 128 |
| convolution | 256 | 3 × 3 | [1, 1] | same | 256 × 256 |
| convolution | 256 | 3 × 3 | [1, 1] | same | 256 × 256 |
| up convolution | 128 | 3 × 3 | [2, 2] | same | 256 x 256 |
| convolution | 128 | 3 × 3 | [1, 1] | same | 256 × 256 |
| convolution | 128 | 3 × 3 | [1, 1] | same | 512 × 512 |
| up convolution | 64 | 3 × 3 | [2, 2] | same | 512 × 512 |
| convolution | 64 | 3 × 3 | [1, 1] | same | 512 × 512 |
| convolution | 64 | 3 × 3 | [1, 1] | same | 512 × 512 |
| convolution | 1 | 1 × 1 | [1, 1] | same | 512 × 512 |

The output of 0-th level from a well-trained model is the desired meta-image, i.e., M⁽¹⁾=^{r}f^{[0]}. Let C^{(L)} be the labeling matrix for C. The loss function L_{UNet} is defined based on the binary cross-entropy loss:

$${ℒ}_{U Net} ( {C}^{( L )} , {M}^{( 1 )} ) = - \frac{1}{w \times h} {\sum }_{i = 1}^{w} {\sum }_{j = 1}^{h} {C}_{i , j}^{( L )} log ( {M}_{i , j}^{( 1 )} ) + ( 1 - {C}_{i , j}^{( L )} ) log ( 1 - {M}_{i , j}^{( 1 )} ) \qquad ( 6 )$$

In a word, this example shows that existing deepnets are sufficient to represent most requested knowledge in a similar manner, instead of always creating new ones for KRs. Also note that a knowledge rule can be implemented by different deepnets. For example, a U-Net variant called SE-U-Net (see Jiang et al., “SE-U-Net: Contextual segmentation by loosely coupled deep networks for medical imaging industry,” ACIIDS, 2021) which is proposed for lung segmentation could be considered as another meta-image generating implementation for KR1.

Meta-images from analytics-based approach. KR2 was used as an example to generate meta-image M⁽²⁾, shown in FIG. 3. The analytics-based function for KR2, called tumor brightness constraint (denoted as K_{TBC}(·)), is to weight the pixels between boundary thresholds b^{⊥} and b^{T} which mostly likely define tumors' brightness region summarized from doctors' experiences. By Eq. (1) the meta-image generating function for KR2 is written as M⁽²⁾=K_{TBC}(C, b^{⊥}, b^{T}), and K_{TBC}(·) can be further represented as follows:

$${M}_{i , j}^{( 2 )} = \{ \begin{matrix}255 , & if {b}^{⊥} \leq {C}_{i , j} \leq {b}^{⊤} , \\ 0 , & otherwise .\end{matrix} \qquad ( 7 )$$

where 1≤i≤w, 1≤j≤h, and the maximum pixel value in a CT image is 255. The analytics-related KRs can be transformed into meta-images in a similar manner.

Once meta-images are obtained, they together with the original CT image can be combined as the knowledge-annotated image A. Assume n meta-images are generated for the CT image C_{w×h}. Then, knowledge-annotated image A is constructed by stacking the CT image and meta-images, expressed as:

$$\begin{matrix}{A}_{w \times h \times ( n + 1 )} = C ⊕ {M}^{( 1 )} ⊕ … ⊕ {M}^{( n )} ( operation corresponding to Fig . 3 ) \\ = tensor ( C , {M}^{( 1 )} , … , {M}^{( n )} ) ( Python - like implementation )\end{matrix} \qquad ( 8 )$$

Although the capacity of deepnets is hard to directly measure from the input tensor and the network structure, calculating the amount of increased feature dimensions is still a referential way to perceive the capacity improvement. For a convolutional deepnet whose first-layer neurons use stride size s, padding size ρ, and kernel size κ×κ, the amount of increased feature dimensions incurred by exotic knowledge in A is calculated as follows (compared to C):

$$n \times ( \frac{( w - κ ) + 2 ρ}{s} \times \frac{( h - κ ) + 2 ρ}{s} ) \qquad ( 9 )$$

The higher the quantity of Eq. (9), the greater the degree of strengthening tumor detection capacity.

### C. Diagnosis Deepnet and Loss Functions Considering Knowledge in Meta-Images

Table II shows the detailed specifications of the diagnosis deepnet, modified from the YOLO [10]. The fundamental components include the convolution layer and the residual layer with different kernels, stride and padding parameters. Certain layers are combined as a block, which is then repeatedly stacked multiple times, as shown in the first column of the table. The diagnosis vector d is produced by the softmax layer with incoming features generated by previous layers.

TABLE II
Network structure of the diagnosis
deep-net (modified from YOLO)

| | | #(Ker- | | | | Output |
|---|---|---|---|---|---|---|
| Times | Type | nels) | Size | Stride | Padding | Size |
| 1x | convolution | 32 | 3 × 3 | [1, 1] | same | 256 × 256 |
| | convolution | 64 | 3 × 3/2 | [2, 2] | same | 128 × 128 |
| | convolution | 32 | 1 × 1 | [1, 1] | same | |
| | convolution | 64 | 3 × 3 | [1, 1] | same | |
| | residual | | | | | 128 × 128 |
| 2x | convolution | 128 | 3 × 3 | [2, 2] | same | 64 × 64 |
| | convolution | 32 | 1 × 1 | [1, 1] | same | |
| | convolution | 64 | 3 × 3 | [1, 1] | same | |
| | residual | | | | | 64 × 64 |
| 8x | convolution | 256 | 3 × 3/2 | [2, 2] | same | 32 × 32 |
| | convolution | 128 | 1 × 1 | [1, 1] | same | |
| | convolution | 256 | 3 × 3 | [1, 1] | same | |
| | residual | | | | | 32 × 32 |
| 8x | convolution | 512 | 3 × 3/2 | [2, 2] | same | 16 × 16 |
| | convolution | 256 | 1 × 1 | [1, 1] | same | |
| | convolution | 512 | 3 × 3 | [1, 1] | same | |
| | residual | | | | | 16 × 16 |
| 4x | convolution | 1024 | 3 × 3/2 | [2, 2] | same | 8 × 8 |
| | convolution | 512 | 1 × 1 | [1, 1] | same | |
| | convolution | 1024 | 3 × 3 | [2, 2] | same | |
| | residual | | | | | 8 × 8 |
| | Avgpool | | Global | | | |
| | Connected | | 1000 | | | |
| | Softmax | | | | | |

Designing loss functions with integrating meta-images is the key to increasing model capacity of the diagnosis deepnet. The loss function, denoted by L_{diagnosis}, of the diagnosis deepnet needs to consider not only the detection loss L_{detection} but also the knowledge loss L_{knowledge} in evaluating diagnosis decisions, and is represented as follows:

[symbol]

_{diagnosis}= [symbol] _{detection}+ [symbol] _{knowledge} (10)

The design concepts inside the loss function give hints as to why the proposed scheme achieves superior performance, presented in detail as follows.

Design of L_{detections}, This loss term minimizes the detection error, which mimics the existing object detection deep nets, such as YOLO, and is calculated as:

[symbol]

_{detection}=λ [symbol] Σᵢ₌₀ [symbol] Σⱼ₌₀ [symbol] ((x_{i,j}−\hat{x}_{i,j})²+(y_{i,j}−ŷ_{i,j})³) +λ [symbol] Σᵢ₌₀ [symbol] Σⱼ₌₀^{B}t_{i,j} [symbol] (( [symbol] − [symbol] )²+( [symbol] − [symbol] )²) +Σᵢ₌₀ [symbol] Σⱼ₌₀ [symbol] (−log(p̅_{i,j})) +λ [symbol] Σᵢ₌₀ [symbol] Σⱼ₌₀ [symbol] (−log(1−p̅_{i,j})) +Σᵢ₌₀ [symbol] Σⱼ₌₀ [symbol] Σ [symbol] (−clog( [symbol] )−(1−c)log(1− [symbol] )) (11)

The notations follow the original definitions in Redmon et al. (“You Only Look Once: Unified, real-time object detection,” in IEEE Conference on Computer Vision and Pattern Recognition (CVPR), pp. 779-788, 2016).

Design of L_{knowledge}, This loss term minimizes knowledge error, and is represented as the sum of diagnosis loss values in various KR views, shown below.

$${ℒ}_{knowledge} = {\underbrace{{ℒ}_{orr}}}_{for KR 1} + {\underbrace{{ℒ}_{brr}}}_{for KR 2} \qquad ( 12 )$$

where Lₒᵣᵣ and L_{brr} are the loss terms corresponding to KR1 and KR2, respectively, in this study, described as follows.

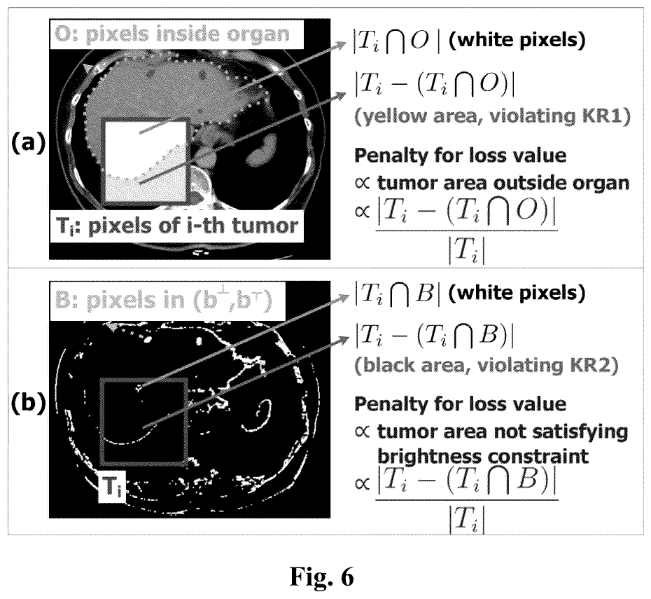

FIG. 6(a) illustrates the loss term design of organ region restriction Lₒᵣᵣ, which is derived from KR1. Let O be the pixel set of the organ region in CT image C and Tᵢ, i=1, . . . , N, be the pixel set of i-th tumor in C. By our proposed scheme, O and Tᵢ can be expressed as

O={C_{u,v}|1≤u≤w,1≤v≤h,M_{u,v}⁽¹⁾>0.} (13)

Tᵢ={C_{u,v}|1≤u≤w,1≤v≤h,(u,v)∈tmrarea(i).} (14)

where tmrarea(i) is the i-th tumor area indicated in diagnosis vector d. Since a tumor area in d is represented by a rectangle, the horizontal and vertical ranges of tmrarea(i) are

$$[ {\hat{x}}_{i} - \frac{{\hat{w}}_{i}}{2} , {\hat{x}}_{i} - \frac{{\hat{w}}_{i}}{2} ] and [ {\hat{y}}_{i} - \frac{{\hat{h}}_{i}}{2} , {\hat{y}}_{i} - \frac{{\hat{h}}_{i}}{2} ] ,$$

respectively. By KR1, pixels that belong to an identified tumor and stay outside the organ region (recorded in M⁽¹⁾, referring to Eq. (2)) are penalized. Thus, Lₒᵣᵣ can be calculated as follows:

$$\begin{matrix}{ℒ}_{orr} & = {λ}_{a} {\sum }_{i = 0}^{N} \frac{\vert {T}_{i} - ( {T}_{i} ⋂ O ) \vert}{\vert {T}_{i} \vert} ( - log ( 1 - \hat{p} ) ) ( Ref . to Fig . 6 ( a ) )\end{matrix} \qquad ( 15 )$$

$$= {λ}_{a} {\sum }_{i = 0}^{N} \frac{1}{{\hat{w}}_{i} \times {\hat{h}}_{i}} {\sum }_{u , v ∈ \text{?} area ( i )} {uv}_{out}^{} ( \frac{255 - {M}_{u , v}^{( 1 )}}{255} ) ( - log ( 1 - {\hat{p}}_{uv} ) ) \qquad ( 16 )$$

$$\text{?} \text{indicates text missing or illegible when filed}$$

where |X| indicates that the number of pixels in the set X, \hat{p}ᵤᵥ is the confidence that pixel (u, v) is in a tumor, and 1ᵤᵥ^{out} returns 1 if pixel (u, v) is outside the organ region; 0 otherwise. Eq. (16) is the computational estimation form to Eq. (15) by using diagnosis output d and meta-image M⁽¹⁾.

FIG. 6(b) illustrates the loss term design of brightness restriction _{brr}, which is derived from KR2. Let B be the pixel set whose brightness satisfies KR2, i.e., M⁽²⁾. By the scheme of the present invention, B can be expressed as

B={C_{u,v}|1≤u≤w,1≤v≤h,M_{u,v}⁽²⁾>0.} (17)

By KR2, pixels that belong to an identified tumor and their brightness do not meet the brightness range between b^{⊥} and b^{T} (recorded in M⁽²⁾, referring to Eq. (7)) are penalized. Thus, L_{brr} is calculated as follows:

$$\begin{matrix}{ℒ}_{brr} & = {λ}_{b} {\sum }_{i = 0}^{N} \frac{\vert {T}_{i} - ( {T}_{i} ⋂ B ) \vert}{\vert {T}_{i} \vert} ( - log ( 1 - \hat{p} ) ) ( Ref . to Fig . 6 ( b ) ) \end{matrix} \qquad ( 18 )$$

$$= {λ}_{b} {\sum }_{i = 0}^{N} \frac{1}{{\hat{w}}_{i} \times {\hat{h}}_{i}} {\sum }_{u , v ∈ \text{?} area ( i )} {uv}_{out}^{} ( \frac{255 - {M}_{u , v}^{( 2 )}}{255} ) ( - log ( 1 - {\hat{p}}_{uv} ) ) \qquad ( 19 )$$

$$\text{?} \text{indicates text missing or illegible when filed}$$

Eq. (19) is the computational estimation form to Eq. (18) by using diagnosis output d and meta-image M⁽²⁾.

All in all, meta-images successfully provide a uniform way to enrich semantics of CT images from different knowledge views, referring to Eqs. (2, 7, 8). Merging all meta-images to the knowledge loss L_{knowledge} for the diagnosis deepnet is elaborated upon in the calculation of Lₒᵣᵣ and L_{brr}, as shown in Eqs. (12, 15, 18). Then, the optimizer is able to tune parameters that do not meet exotic knowledge via L_{knowledge} during the model creation stage. Hence, the meta-image-based diagnosis deepnets have a greater chance of obtaining effective models.

### D. Discussion: What if Low-Quality Knowledge is Encoded?

Notice that the essence of the proposed scheme is to encode the expert knowledge into inputs of the deepnets for improving prediction. Different outcomes might arise if low-quality knowledge is encoded, and human beings often make mistakes. On one hand, once low-quality knowledge is adopted and transformed into the knowledge-annotated image, then intuitively, the meta-images from low-quality knowledge may incur low-quality features inside the deepnet, which will act like noises in model creation and inference. Moreover, meta-images from low-quality knowledge also affect estimation of L_{knowledge}, which leads to less appropriate decisions for neuron parameters during the training stage. Thus, noises and biased loss estimation brings a less positive impact in creating models. On the other hand, the data labels provide another force to determine proper neuron weights via L_{detection} during the training stage. Factoring in the influence from the above two aspects based on our experience, prediction performance of deepnets with low-quality knowledge would be degraded, compared to that with high-quality knowledge, but inference could still be sufficiently accurate when few noises are encountered. That is, the scheme of the present invention may achieve acceptable performance, even when some low-quality knowledge is encoded. The experiments designed to illustrate the scheme are provided in the following examples.

### EXAMPLES

Case Study

System Deployment and Experimental Settings

The prototype of the meta-image-based tumor detection system of the present invention is described in previous sections with Python and PyTorch. The experiments are performed on a Linux-based computer with 2.90 GHz CPU of 12 cores, 16 GB RAM, and a GPU card of NVIDIA GTX 3090 Ti. The deepnet architectures used in the KR1 transformation and the diagnosis deepnet are shown in Tables I and II, respectively. For KR2, the brightness region [b^{⊥}, b^{T}] is set to [40, 150]. The experimental data comes from our academic-industrial cooperation with a hospital of central Taiwan. The dataset contains 400 CT images wherein patients used no contrast agent before the CT-scanning process. Doctors of the hospital assisted in labeling the CT images, such that experimental results are close to real-world scenarios. The ratio of the training/validation/testing data is 0.8:0.1:0.1. The compared deepnets include YOLO, SSD (see Liu et al., “SSD: Single shot multibox detector,” in European Conference on Computer Vision. pp. 21-37, Springer, 2016), and Faster-RCNN (see Ren et al., “Faster R-CNN: Towards real-time object detection with region proposal networks,” Advances in Neural Information Processing Systems, Vol. 28, 2015). The scheme of the present invention was implemented on both YOLO and Faster-RCNN network backbones for experiments. The performance metrics contain precision (PCS), recall (RCL), F1 score (F1), and mean average precision (mAP), which are widely used in the classification and detection works.

### Example 1: Performance Comparisons and Visualization

For verifying effectiveness, the first experiment is to compare meta-image-based deepnets to existing ones in different performance metrics, and Table III shows the comparison results in three groups.

TABLE III
Performance
comparisons for proposed meta-images with different deepnets.

| deep network | PCS | RCL | mAP | F1 | $\# ( \begin{matrix}improved \\ metrics\end{matrix} )$ |
|---|---|---|---|---|---|
| YOLO [13] | 0.54 | 0.55 | 0.42 | 0.54 | — |
| SSD [20] | 0.56 | 0.58 | 0.46 | 0.57 | — |
| Faster-RCNN | 0.46 | 0.62 | 0.52 | 0.53 | — |
| [23] | | | | | |
| KR1@YOLO | 0.66 | 0.62 | 0.57 | 0.64 | 4 |
| KR2@YOLO | 0.62 | 0.64 | 0.55 | 0.63 | 4 |
| KR1 + KR2@ | 0.65 | 0.66 | 0.59 | 0.65 | 4 |
| YOLO | | | | | |
| KR1@Faster- | 0.56 | 0.62 | 0.57 | 0.59 | 3 |
| RCNN | | | | | |
| KR2@Faster- | 0.53 | 0.65 | 0.56 | 0.58 | 4 |
| RCNN | | | | | |
| KR1 + KR2@ | 0.55 | 0.66 | 0.58 | 0.60 | 4 |
| Faster-RCNN | | | | | |

The first group shows the experimental results of existing deepnets, which provide baselines in testing the used dataset. In the second group, three meta-images are applied to the YOLO, denoted as KR1@YOLO, KR2@YOLO, KR1+KR2@YOLO, respectively. From the results, all three of the meta-image-based YOLO networks significantly improve YOLO in all four metrics by 20% on average. The KR1+KR2@YOLO performs best in the first two groups, indicating the KR-based deepnets successfully increase the tumor detection capacity by using transformed exotic knowledge. In the third group, three meta-images are applied to the Fast-RCNN, and results are similar to trends in the second group. This shows that the meta-image mechanism performs robustly with different deepnet backbones.

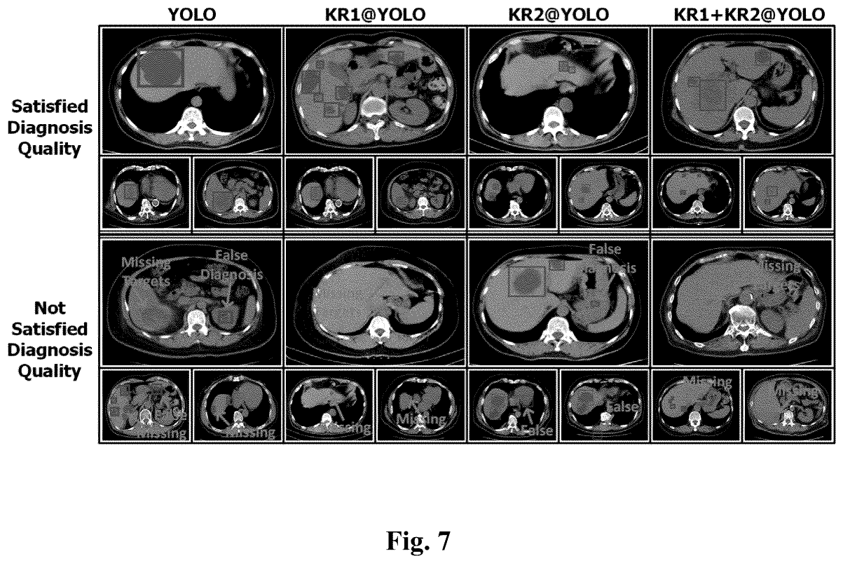

FIG. 7 visualizes some satisfied and unsatisfied cases in different meta-images with the YOLO backbone. In general, the meta-image-based deepnets successfully detect tumors smaller than YOLO. Recall that KR1 describes the organ boundary constraint, and thus false diagnoses outside the target organ (e.g., the unsatisfied case of KR2) are greatly reduced in KR1@YOLO. On the contrary, KR2 describes the brightness constraint, and thus missing targets due to a small region of similar grey levels (e.g., the unsatisfied cases of KR1 and YOLO) are remarkably alleviated in KR2@YOLO. While both KRs are adopted simultaneously (i.e., KR1+KR2), many unsatisfied cases are avoided and the tumor detection capacity is significantly improved. The middle tumor in the satisfied case of KR1+KR2@YOLO is a successful instance, whereas it is missed in previous KR-based deepnets. The above observations are consistent with statistical results in Table III and provide visual evidence for efficacy of meta-images. The unsatisfied case of KR1+KR2 happens in vague and indistinct tumors, which are also easily overlooked even by doctors.

### Example 2: Effect of Low-Quality Knowledge

The experiment studies the effect of the exotic knowledge transformation in the proposed deepnet pipeline in FIG. 3. For ease of study, we degrade K_{U-Net}(·) and K_{TBC}(·) to generate two meta-images for mimicking low-quality knowledge: M_{LQ}⁽¹⁾ for low-quality organ boundary (only half of organ region) and M_{LQ}⁽²⁾ for low-quality brightness boundary (over highlighting moisture effect, setting [b^{⊥}, b^{T}]=[30, 59]). Table IV shows the results of low-quality meta-images used in our proposed scheme.

TABLE IV
Performance comparisons for meta-images of low
qualities with the uniform YOLO backbone.

| deep network | PCS | RCL | mAP | F1 | dist_{YOLO}(•) |
|---|---|---|---|---|---|
| YOLO [13] | 0.54 | 0.55 | 0.42 | 0.54 | — |
| KR1@YOLO | 0.66 | 0.62 | 0.57 | 0.64 | 0.23 |
| M_{LQ}⁽¹⁾ @ YOLO | 0.52 | 0.40 | 0.35 | 0.45 | 0.19 |
| KR2@YOLO | 0.62 | 0.64 | 0.55 | 0.63 | 0.20 |
| M_{LQ}⁽²⁾ @ YOLO | 0.50 | 0.45 | 0.34 | 0.47 | 0.15 |
| KR1 + KR2@YOLO | 0.65 | 0.66 | 0.59 | 0.65 | 0.26 |
| M_{LQ}⁽¹⁾ + M_{LQ}⁽²⁾ @YOLO | 0.51 | 0.38 | 0.32 | 0.44 | 0.22 |

Two facts are observed from the results. Firstly, as expected, the low-quality knowledge (i.e., M_{LQ}⁽¹⁾ and M_{LQ}⁽²⁾) results in lower performance compared with high-quality knowledge (i.e., KR1 and KR2), as seen in the rows highlighted in gray in each group of the table. This shows the efficacy of the meta-image model from a knowledge quality perspective.

Secondly, comparing the gray-highlighted rows to the first one, the performance achieved by low-quality knowledge is decreased compared with that of YOLO. For quantitatively expressing the concept, distances of performance vectors were used to measure the similarity between KR-based methods and YOLO:

$${dist}_{y} ( x ) = {‖ ( \begin{matrix}PCS ( x ) - PCS ( y ) , RCL ( x ) - RCL ( y ) , \\ mAP ( x ) - mAP ( y ) , F 1 ( x ) - F 1 ( y )\end{matrix} ) ‖}_{2} \qquad ( 20 )$$

where x and y indicate a KR-based method and YOLO, respectively, shown in the last column of the table. The results show that performance of low-quality knowledge is close to YOLO, compared to that of high-quality knowledge. Recall that the loss L_{diagnosis} consists of L_{detection} and L_{knowledge} in Eq. (10). While low-quality knowledge declines model capacity via L_{knowledge}, the optimizer still produces rudimentary capacity with labeled data via L_{detection}. In sum, the performance degrades to that of the YOLO's level.

### Example 3: Properties of Creating Diagnosis Deepnet Models

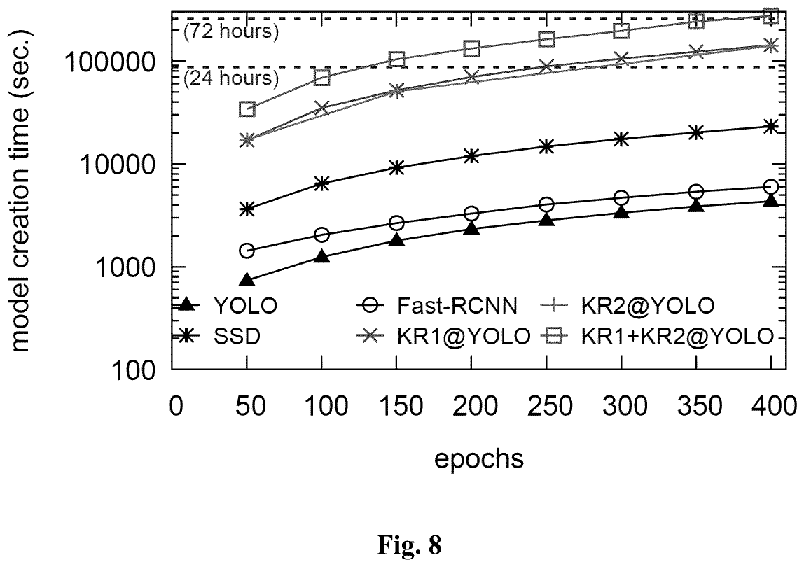

The last experiment studies the model creation time and model capacity of the diagnosis deepnets. FIG. 8 shows the time cost of creating diagnosis models for various deepnets in different sizes of the training dataset. From the results, meta-image-based deepnets (marked with prefix KR-x in the figure) require more training time than others, which shows that our scheme exchanges a high training-time cost for high diagnosis capacity. The high training-time cost, mostly between 1-3 days, comes from calculation of the loss L_{knowledge}, as it needs to evaluate every pixel in tumor areas, referring to Eqs. (15-19). Since diagnosis performance is indeed improved, such time costs are acceptable for most hospitals due to the industrial benefits.

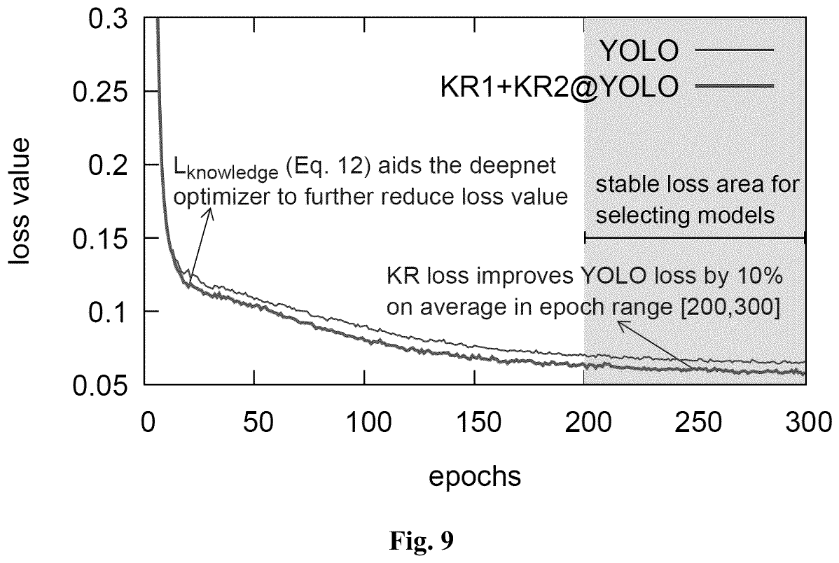

FIG. 9 shows the loss value as a function of epochs in the training stage for KR1+KR2@YOLO and YOLO. After 20 epochs, the developed loss function (KR1+KR2@YOLO) already obtains lower loss values than YOLO. Furthermore, in the stable loss area, i.e., epoch period [200, 300], the scheme of the present invention notably improves loss values by 10% on average. The results provide evidence that the deepnet pipeline of the present invention can create models of high tumor detection capacity.

### CONCLUSIONS

In the present invention, a novel meta-image-based tumor detection deepnet pipeline is created to provide accurate diagnosis services for increasing medical quality. The core function is the meta-image model which uniformly transforms medical images to knowledge-embedded tensors for deepnets. The generated meta-images together with the knowledge-derived loss functions aid the deepnet optimizer to create powerful tumor detection models. For verifying effectiveness of the scheme of the present invention, real-world experiments were conducted on abdominal CT images from different evaluation perspectives: exemplary KRs (KR1 and KR2), low-quality KRs, and model training properties. Testing results of all perspectives explicitly show that the scheme of the present invention achieves superior performance than existing methods by 20% in different metrics. The developed prototype has been internally examined by doctors in a hospital of central Taiwan, showing the practicability of the scheme of the present invention.

### REFERENCES

[1] A. Hosny, C. Parmar, J. Quackenbush, L. H. Schwartz, and H. J. Aerts, “Artificial intelligence in radiology,” Nature Reviews Cancer, vol. 18, no. 8, pp. 500-510, 2018.
[2] L. Song, H. Wang, and Z. J. Wang, “Bridging the gap between 2D and 3D contexts in CT volume for liver and tumor segmentation,” IEEE Journal of Biomedical and Health Informatics, vol. 25, no. 9, pp. 3450-3459, 2021.
[3] T. Bai, B. Wang, D. Nguyen, B. Wang, B. Dong, W. Cong, M. K. Kalra, and S. Jiang, “Deep interactive denoiser (DID) for X-ray computed tomography,” IEEE Transactions on Medical Imaging, vol. 40, no. 11, pp. 2965-2975, 2021.
[4] M. Matsuura, J. Zhou, N. Akino, and Z. Yu, “Feature-aware deep-learning reconstruction for context-sensitive X-ray computed tomography,”IEEE Transactions on Radiation and Plasma Medical Sciences, vol. 5, no. 1, pp. 99-107, 2021.
[5] C. Zhao, M. Shen, L. Sun, and G.-Z. Yang, “Generative localization with uncertainty estimation through video-CT data for bronchoscopic biopsy,” IEEE Robotics and Automation Letters, vol. 5, no. 1, pp. 258-265, 2020.
[6] H. Ren, T. Li, and Y. Pang, “A fully automatic framework to localize esophageal tumor for radiation therapy,” in IEEE International Conference on Real-time Computing and Robotics (RCAR), 2019, pp. 510-515.
[7] X.-Y. Zhou, J.-Q. Zheng, P. Li, and G.-Z. Yang, “ACNN: a full resolution DCNN for medical image segmentation,” in IEEE International Conference on Robotics and Automation (ICRA), 2020, pp. 8455-8461.
[8] Z. Lin, Z. He, S. Xie, X. Wang, J. Tan, J. Lu, and B. Tan, “AANet: Adaptive attention network for COVID-19 detection from chest X-ray images,” IEEE Transactions on Neural Networks and Learning Systems, vol. 32, no. 11, pp. 4781-4792, 2021.
[9] Y. Xie, Y. Xia, J. Zhang, Y. Song, D. Feng, M. Fulham, and W. Cai, “Knowledge-based collaborative deep learning for benign-malignant lung nodule classification on chest CT,” IEEE Transactions on Medical Imaging, vol. 38, no. 4, pp. 991-1004, 2019.
[10] J. Redmon, S. K. Divvala, R. B. Girshick, and A. Farhadi, “You Only Look Once: Unified, real-time object detection,” in IEEE Conference on Computer Vision and Pattern Recognition (CVPR), 2016, pp. 779-788.
[11] O. Ronneberger, P. Fischer, and T. Brox, “U-Net: Convolutional net-works for biomedical image segmentation,” in International Conference on Medical Image Computing and Computer-Assisted Intervention. Springer, 2015, pp. 234-241.
[12] L.-Y. Jiang, C.-J. Kuo, T.-H. O, M.-H. Hung, and C.-C. Chen, “SE-U-Net: Contextual segmentation by loosely coupled deep networks for medical imaging industry,” in ACIIDS, 2021.
[13] W. Liu, D. Anguelov, D. Erhan, C. Szegedy, S. Reed, C.-Y. Fu, and A. C. Berg, “SSD: Single shot multibox detector,” in European Conference on Computer Vision. Springer, 2016, pp. 21-37.
[14] S. Ren, K. He, R. Girshick, and J. Sun, “Faster R-CNN: Towards real-time object detection with region proposal networks,” Advances in Neural Information Processing Systems, vol. 28, 2015.

What is claimed is:

1. A method for diagnosing tumors on a medical image, wherein the diagnosis is made through analyzing the medical image by a diagnosis model, wherein the diagnosis model comprises meta-image-based deepnets developed by cooperating with experts' knowledge for accurate tumor recognition in medical images.

2. The method of claim 1, wherein the method comprises creating a diagnosis model with integrating adopted knowledge to design appropriate loss functions by counting loss values occurring in meta-images during training a tumor detection deepnet.

3. The method of claim 2, wherein the meta-images are generated from transforming knowledge rules by a deepnet-based approach and/or an analytics-based approach.

4. The method of claim 3, wherein the deepnet-based approach comprises using deepnets to represent knowledge rules and constructing a meta-image.

5. The method of claim 3, wherein the analytics-based approach comprising using analytic models to find pixels of medical images that fit the brightness range and constructing a meta-image.

6. The method of claim 3, wherein meta-image is created by uniformly transforming medical images to knowledge-embedded tensors for a deepnet and improving the deepnet capacity by increasing the dimension of feature space from exotic domain knowledge.

7. The method of claim 1, wherein the medical image is obtained from an imaging technology selected from X-ray radiography, magnetic resonance imaging, ultrasound, endoscopy, elastography, tactile imaging, thermography, medical photography, positron emission tomography (PET) and computed tomography (CT).

8. The method of claim 3, wherein the knowledge rules include determining the organ region, identifying the tumors that reside in the organ region, and displaying the tumors in a specified brightness range.

9. The method of claim 8, wherein the knowledge rules are translated by the hybrid of the deepnet-based and analytics-based approaches, and wherein human knowledge and image data are mixed in the image format.

10. The method of claim 2, wherein the loss functions are knowledge-derived loss functions that aid a deepnet optimizer to create powerful tumor detection models.

11. The method of claim 10, wherein the optimizer is used to tune parameters that do not meet exotic knowledge via loss function of knowledge during the model creation stage.

12. The method of claim 1, wherein the tumor is selected from bladder tumors, breast tumors, cervical tumors, colon or rectal tumors, endometrial tumors, kidney tumors, lip or oral tumors, liver tumors, skin tumors, lung tumors, ovarian tumors, pancreatic tumors, prostate tumors, thyroid tumors, brain tumors, bone tumors, muscle or tendon tumor, tumors of the nervous system, and tumors of the gastrointestinal system.

13. A method for processing a medical image, comprising generating meta-images from transforming knowledge rules by the deepnet-based approach and/or the analytics-based approach.

14. The method of claim 13, wherein the deepnet-based approach comprises using deepnets to represent knowledge rules and constructing a meta-image.

15. The method of claim 13, wherein the analytics-based approach comprises using analytic models to find pixels of medical images that fit the brightness range and constructing a meta-image.

16. The method of claim 13, wherein meta-image is created by uniformly transforming medical images to knowledge-embedded tensors for deepnet and improving the deepnet capacity by increasing the dimension of feature space from exotic domain knowledge.

17. The method of claim 13, wherein the medical image is selected from CT images and X-ray images.

18. The method of claim 13, wherein the knowledge rules include determining the organ region, identifying the tumors that reside in the organ region, and displaying the tumors in a specified brightness range.

19. The method of claim 18, wherein the knowledge rules are translated by the hybrid of the deepnet-based and analytics-based approaches, and wherein human knowledge and image data are mixed in the image format.

20. The method of claim 16, wherein a deepnet optimizer is used to tune parameters that do not meet exotic knowledge via loss function of knowledge during the model creation stage.
